(19) United States
(12) Patent Application Publication
(10) Pub. No.: US 2004/0115139 A1
(43) Pub. Date: Jun. 17, 2004

(54) Botulinum toxin dental therapies and procedures

(75) Inventors: Howard I. Katz, La Jolla, CA (US); Andrew M. Blumenfeld, San Diego, CA (US)

Correspondence Address: STEPHEN DONOVAN, ALLERGAN, INC., 2525 Dupont Drive, T2-7H, Irvine, CA, 92612, US

(73) Assignee: Allergan, Inc.

(21) Appl. No.: 10/685,986

(22) Filed: Oct. 15, 2003

Related U.S. Application Data
(60) Provisional application No. 60/418,789, filed on Oct. 15, 2002.

(51) Int. Cl. A61K 7/28; A61C 5/00

(52) U.S. Cl. 424/50; 433/217.1

(57) ABSTRACT

Methods for facilitating various dental treatments, therapies and procedures by administration of a Clostridial toxin to a muscle or other tissue on the head or neck of a patient.

### CROSS REFERENCE

This application claims priority to provisional application serial No. 60/418,789, filed Oct. 15, 2002, the entire content of which is incorporated herein.

### BACKGROUND

The present invention relates to use of a Clostridial toxin in dentistry. In particular the present invention relates to use of a botulinum toxin in conjunction with a dental therapy, treatment or procedure.

Social and physical well being is related to the presence of teeth in an individual's mouth. Teeth permit food to be chewed, a pleasant smile to be made and maintenance of a normal facial appearance. Keeping functioning teeth in the mouth of an individual can depend upon adherence of healthy gum tissue to the teeth as well as to proper positioning and seating of the teeth in surrounding bone.

Many dental therapies, treatment and procedures can be carried out upon a tooth or upon gum tissue, such as a tooth implant, a tooth transplant, and gum tissue transplantation. Additionally, there are numerous dental repair or restoration procedures which include placement of a dental artifact, such as a crown, veneer, splint, brace, filling, etc into the mouth of a patient. Teeth, gum tissue, bone and dental restorative and cosmetic materials (dental artifacts) can be affected by a force applied to them by a chewing or mastication muscle. The mastication muscles are the masseter, temporalis, lateral (external) pterygoid and medial (internal) pterygoid muscles. The force exerted by a mastication muscle upon a tooth, an oral tissue or upon a dental artifact can be deleterious to a dental therapy, treatment or procedure. Additionally, a patient with a misaligned jaw or with a parafunctional habit can subject his teeth, periodontal ligament, alveolar bone, muscle attachments, and temperomandibular joint to a mastication muscle force which can cause a dental treatment, therapy or procedure to be unsuccessful.

The hindrance to a dental treatment, therapy or procedure, including a dental repair or restoration procedure, by the presence of a force, such as a bite force applied by a mastication muscle in the mouth at the location or in the vicinity of the location of a dental procedure can result in a loosening of a tooth or of a dental artifact and/or to a reduced lifespan of the tooth or dental artifact in the mouth of the patient. Such a loosening or reduced life span of a tooth or of a dental artifact can occur due to suboptimal adherence of the tooth or dental artifact to a substrate in the mouth of the patient.

Unfortunately, attempts to address a deleterious affect of a mastication muscle force upon a tooth, gum tissue or upon a dental artifact in the mouth of a patient have been unsuccessful or have significant drawbacks and deficiencies. Thus, there is typically a low patient compliance rate to use of a protective removable appliance worn over the teeth. Furthermore, typical treatment for an oral muscle pain and a related facial joint pain by administration of an analgesic or anti-inflammatory compound can have deleterious local and systemic effects.

Botulinum Toxin

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex)¹ is a LD50 in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD50 of botulinum toxin type A of 0.3 ng equals 1 U is corrected for the fact that about 0.05 ng of BOTOX® equals 1 unit). One unit (U) of botulinum toxin is defined as the LD50 upon intraperitoneal injection into female Swiss Webster mice weighing 18 to 20 grams each. Available from Allergan, Inc., of Irvine, Calif. under the tradename BOTOX® in 100 unit vials)

Seven generally immunologically distinct botulinum neurotoxins have been characterized, these being respectively botulinum neurotoxin serotypes A, B, C1, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD50 for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine. Additional uptake can take place through low affinity receptors, as well as by phagocytosis and pinocytosis.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, HC, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, HN, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C1 was originally thought to cleave syntaxin, but was found to cleave syntaxin and SNAP-25. Each of the botulinum toxins specifically cleaves a different bond, except botulinum toxin type B (and tetanus toxin) which cleave the same bond. Each of these cleavages block the process of vesicle-membrane docking, thereby preventing exocytosis of vesicle content.

Botulinum toxins have been used in clinical settings for the treatment of neuromuscular disorders characterized by hyperactive skeletal muscles (i.e. motor disorders). In 1989 a botulinum toxin type A complex has been approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C1 has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem J 1;339 (pt 1):159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C1 is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9);1373-1412 at 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [3H]Noradrenaline and [3H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C1, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×107 U/mg, an A260/A278 of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×108 LD50 U/mg or greater; purified botulinum toxin type B with an approximately 156 kD molecular weight with a specific potency of 1-2×108 LD50 U/mg or greater, and; purified botulinum toxin type F with an approximately 155 kD molecular weight with a specific potency of 1-2×107 LD50 U/mg or greater.

Botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying.

The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

It has been reported that botulinum toxin type A has been used in clinical settings as follows:

(1) about 75-125 units of BOTOX® per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX(® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).

(6) to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U

(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

It is known that botulinum toxin type A can have an efficacy for up to 12 months (European J. Neurology 6 (Supp 4): S111-S1150:1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996;114(3):507, and The Laryngoscope 109:1344-1346:1999. However, the usual duration of effect of an intramuscular injection of Botox® is typically about 3 to 4 months.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A Botulinum toxin type B preparation (MyoBloc®) is available from Elan Pharmaceuticals of San Francisco, Calif.

In addition to having pharmacologic actions at the peripheral location, botulinum toxins may also have inhibitory effects in the central nervous system. Work by Weigand et al, Nauny-Schmiedeberg's Arch. Pharmacol. 1976; 292,161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

U.S. Pat. No. 5,989,545 discloses that a modified clostridial neurotoxin or fragment thereof, preferably a botulinum toxin, chemically conjugated or recombinantly fused to a particular targeting moiety can be used to treat pain by administration of the agent to the spinal cord.

A botulinum toxin has also been proposed for the treatment of otitis media of the ear (U.S. Pat. No. 5,766,605), inner ear disorders (U.S. Pat. Nos. 6,265, 379; 6,358,926), tension headache, (U.S. Pat. No. 6,458,365), migraine headache (U.S. Pat. No. 5,714,468), post-operative pain and visceral pain (U.S. Pat. No. 6,464,986), hair growth and hair retention (U.S. Pat. No. 6,299,893), psoriasis and dermatitis (U.S. Pat. No. 5,670,484), injured muscles (U.S. Pat. No. 6,423,319) various cancers (U.S. Pat. No. 6,139,845), smooth muscle disorders (U.S. Pat. No. 5,437,291), and neurogenic inflammation (U.S. Pat. No. 6,063,768). Controlled release toxin implants are known (see e.g. U.S. Pat. Nos. 6,306,423 and 6,312,708) as is transdermal botulinum toxin administration (U.S. patent application Ser. No. 10/194805).

Periocular injections of a botulinum toxin to treat various eye spasms, such as blepharospasm and strabismus is well known. See e.g. Scott A., Botulinum toxin injection into extraocular muscles as an alternative to strabismus surgery, Ophthalmology 1980;87(10):1044-9. U.S. Pat. No. 5,298,019 discusses use of a botulinum toxin to reduce tooth wear due to involuntary clenching of the mastication muscles. See also, Van Zandijcke M., et al., Treatment of bruxism with botulinum toxin injections, J Neurol Neurosurg Psychiatry 1990;53(6):530, and; Fross R., Bruxism and masticatory myalgias: Use of botulinum toxin, Mov Disord 2000;15(Suppl 2):35. Temporomandibular joint disorders and dislocation have been treated with a botulinum toxin. Schwartz M., et al., Treatment of temporomandibular disorders with botulinum toxin, Clin J Pain 2002;18(6 Suppl):S198-S203. A botulinum toxin has been injected into a head area to treat depression (DE 101 50 415). Palate tremor has been treated by injecting a botulinum toxin into a tensor veli palatini muscle. Deuschl G., et al., Botulinum toxin treatment of palatal tremor (myoclonus), In: Jankovic J, ed. Neurological Disease and Therapy. Therapy with botulinum toxin New York: Marcel Dekker;1994;25:pp. 567-76. Drooling has been treated by injecting a botulinum toxin into a salivary gland. Maik, E., et al., Up-to-date report of botulinum toxin therapy in patients with drooling caused by different etiologies, J Oral Maxillofac Surg 2003 April;61(4):454-457. A botulinum toxin has been injected into the throat, neck, and/or larynx to treat dysphonia, certain swallowing disorders and cervical dystonia. Boutsen F., et al, Botox treatment in adductor spasmodic dysphonia: A meta-analysis, J Speech Lang Hear Res 2002;45(3):469-481, and; Carruthers J., et al (1996), Botulinum A Exotoxin Use in Clinical Dermatology; J Amer Acad Derm 34: 788-797), and; Carruthers J., et al., (1996) Botulinum A Exotoxin in Clinical Ophthalmology; Can. J. Ophthalmol. 31: 389-400).

Facial synkinesis and asymmetry caused by facial nerve palsy have been treated with a botulinum toxin. Armstrong M. et al. (1996) Treatment of Facial Synkinesis and Facial Asymmetry with Botulinum Toxin Type A Following Nerve Palsy, Clin. Otolaryngol. 21:15-20).

A botulinum toxin has been used cosmetically to treat various facial wrinkles. See e.g.. Carruthers J., et al., (1992) Treatment of Glabellar Frown Lines with C. Botulinum-A Exotoxin; J. Dermatol. Surge Oncol. 18: 17-21, and; U.S. Pat. No. 6,358,917.

A botulinum toxin has also been used to treat neck lines upon injection into the platysma muscle. Brandt F., et al., (1998) Cosmetic Use of Botulinum A Exotoxin for the Aging Neck; Dermatol. Surg. 24: 1232-1234).

Injection of a botulinum toxin into the point of the chin has also been done for treatment of prominent mental crease. Carruthers A., et al., Cosmetic Uses of Botulinum A Exotoxin; in: James, W. D. et al Eds. Advances in Dermatology (1997) Mosby-Yearbook, Chicago.

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively).

Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16);9153-9158:1990.

Acetylcholine

Typically only a single type of small molecule neurotransmitter is released by each type of neuron in the mammalian nervous system, although there is evidence which suggests that several neuromodulators can be released by the same neuron. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

What is needed therefore is a method for reducing tooth wear and tooth loss, facilitating positioning, maintaining and reduced wear upon dental artifacts, assisting adherence of oral and gum tissue and teeth (both natural and transplanted), and reducing damage to and loss of oral bone (including loss of oral bone density).

What is also needed is an effective way to prevent oral damage and to enhance treatment to dental hard and soft tissue and restorations by de-programming the muscles responsible for such damage.

### SUMMARY

The present invention provides a method for reducing tooth wear and tooth loss, facilitating positioning, maintaining and reduced wear upon dental artifacts, assisting adherence of oral and gum tissue and teeth (both natural and transplanted), increasing tolerance to dental artifacts, such as dental appliances and dental hardware, and for reducing damage to and loss of oral bone (including loss of oral bone density). The present method also provides an effective way for preventing damage to an oral structure or tissue, and to enhance treatment to dental hard and soft tissue and restorations by de-programming the muscles responsible for such damage.

Our invention includes a method for using a Clostridial neurotoxin, such as a botulinum toxin, to weaken or paralyze a muscle innervated by trigeminal and facial nerves in a patient, such as a mastication muscle, to thereby reduce and/or prevent damage to teeth, gums, periodontal ligaments, alveolar bone, dental restorative materials and/or to the tempero mandibular joint. A method within the scope of our invention can be carried out by; (a) locating a muscle of mastication in the mouth of a patient; (b) measuring a bite force (i.e. with a bite pressure sensor [e.g. Tekscan, gnathomamometer, strain gauges]) or measuring the indentations made by the teeth cusps when the patient bites with maximum force into a suitable (i.e. wood or plastic) material with a calibrated resistance to deformity and fixed dimensions, and; (c) administering (as by injecting) into a muscle which can apply a force which hinders a dental therapy, treatment or procedure with a botulinum toxin sufficient to cause reduction in force which can be exerted by the hindering mastication muscle. Our method can also be used to facilitate healing with or after a dental procedure, by reducing a mastication muscle force.

Thus, practise of the disclosed method results in a reduction in a force applied to oral tissue and to a dental restorative material in the mouth of a patient. The force applied can be re-measured (as set forth in the paragraph above) after the injection of the botulinum toxin, and resulting weakening or paralysis of the muscle, and the decrease of an exerted mastication muscle force thereby quantitated.

The botulinum toxin can be injected into symmetric muscles on opposite sides of the face during the same treatment session (i.e. bilateral injections).

Thus, our invention encompasses use of a botulinum neurotoxin to cause a limited paralysis of one or more of the muscles of mastication in a patient. It is often difficult and uncomfortable for the patient to accommodate to any change in occlusion or posture of the mandible while undergoing a dental treatment or when a dental treatment is complete. These changes can be more easily tolerated, accepted and accommodated by deprogramming the muscles that contribute to discomfort by a method disclosed herein.

Administration of a botulinum neurotoxins to a muscle of the gnathological system of a patient can enhance the tolerance and compliance of a dental therapy and treatment. The botulinum neurotoxin can be used alone or in combination with a fixed or removable appliance or with a dental artifact that changes the occlusion and the vertical or antero-posterior position of the mandible.

Our invention provides a method for preventing damage and augmenting treatment to the teeth, gums, periodontal ligaments, alveolar bone, dental restorative materials, the tempero mandibular joint by first locating a muscle of mastication of a patient. The second step is to determine a neuromuscular ideal position. This can be carried out by measuring a bite force and the ability of the patient to maintain a constant force with a bite pressure sensor (Tekscan, gnathomamometer, strain gauges, EMGs, Computerized Mandibular Positioners, load sensors) or measuring the indentations made by the teeth cusps when the said subject bites with maximum force into a piece of plastic materials with calibrated resistance to deformity of fixed dimensions. The third step is to administer to a mastication muscle (which muscle applies a force such that the neuromuscular ideal position is not attained) a quantity of a botulinum toxin sufficient to reduce a force exerted by that muscle. The resulting reduced force applied by the muscle to an oral tissue and/or to any dental restorative material (i.e. a dental artifact) present in the mouth permits attainment of the neuromuscular ideal position.

A method within the scope of our invention is a method for assisting a dental procedure by: (a) administering a botulinum toxin to a mastication muscle of a patient; (b) waiting a period of time sufficient for the botulinum toxin to weaken the mastication muscle, so that a force which can be applied by the mastication muscle upon a location where a dental procedure will be conducted in the mouth of the patient is reduced, as compared to a force which can be applied by the mastication muscle at the location of the dental procedure prior to administration of the botulinum toxin, and; (c) conducting the dental procedure upon the patient.

The dental procedure can be a tooth implantation, a gum tissue transplantation or a dental cosmetic or restoration procedure. The botulinum toxin can be selected from the group consisting of botulinum toxin types A, B, C, D, E, F and G. Preferably, the botulinum toxin is a botulinum toxin type A. The botulinum toxin can be administered in an amount of between about 1 unit and about 10,000 units. Thus, 10 units to 200 units of the botulinum toxin type A known as BOTOX® can be used, 40 to 800 units of a botulinum toxin type A known as DYSPORT® can be used and 500 to 10,000 units of a botulinum toxin type B known as MYOBLOC® can be used. Additionally, other botulinum toxin serotypes, such as botulinum toxin types E or F, can be used when a short acting or alternate botulinum toxin is desired.

The period of time one waits for it to be sufficient for the botulinum toxin to weaken the mastication muscle can be between about one hour and about thirty days. As should be clear, the dental procedure is conducted or carried out within the mouth of the patient.

Use of a botulinum toxin as set forth herein can assist a dental procedure by facilitating, encouraging or causing an adherence and tolerance of a dental item (which is synonymous with a dental artifact) to a substrate in the mouth of the patient. Adherence means formation of a bond sufficient to securely hold the dental item in the mouth of the patient. Such a bond can be formed by integration of the dental item with adjacent bone (i.e. osseo-integration) and/or tissue, and/or by chemical bonding or wedging of the dental item to a substrate. A “substrate” can be a platform, a pin or other dental artifact, or a substrate can be a bone, tooth or tissue present in the mouth and to which the dental item (i.e. a dental artifact) is placed in contact to or with by the dental procedure.

Significantly, the adherence of the dental item occurs in less time, is maintained for a longer period of time, is a stronger adherence and/or occurs with less inflammation, as compared to the adherence of the dental item when a botulinum toxin has not been administered prior to conducting the same dental procedure.

A detailed embodiment of a method within the scope of our invention can comprise a method for facilitating an adherence of a dental item to a substrate in the mouth of a patient by: (a) administering a botulinum toxin to a mastication muscle of a patient; (b) waiting a period of time sufficient for the botulinum toxin to weaken the mastication muscle, so that a force with can be applied by the mastication muscle upon a location where a dental item can be placed in contact with a substrate in the mouth of the patient is reduced, as compared to a force which can be applied by the mastication muscle at the location where a dental item can be placed in contact with a substrate in the mouth of the patient prior to administration of the botulinum toxin; (c) placing a dental item in contact with a substrate in the mouth of the patient, and; (d) observing adherence of the dental item with the substrate in the mouth of the patient, thereby facilitating adherence of the dental item to the substrate in the mouth of the patient.

The following definitions apply herein:

“About” means approximately or nearly and in the context of a numerical value or range set forth herein means ±10% of the numerical value or range recited or claimed.

“Alleviating” means a reduction in the occurrence of a symptom. Thus, alleviating includes some reduction, significant reduction, near total reduction, and total reduction of a symptom. An alleviating effect may not appear clinically for between 1 to 7 days after administration of a Clostridial toxin to a patient.

“Botulinum toxin” means a botulinum neurotoxin as either pure toxin (i.e. about 150 kDa weight molecule) or complex (i.e. 300-900 kDa weight complex), and excludes botulinum toxins which are not neurotoxins such as the cytotoxic botulinum toxins C2 and C3, but includes recombinantly made, hybrid, modified, and chimeric botulinum toxins.

“Local administration” or “locally administering” means administration (i.e. by a subcutaneous, intramuscular, subdermal or transdermal route) of a pharmaceutical agent to or to the vicinity of a subdermal location or in the head or neck of a patient.

“Treating” means to alleviate (or to eliminate) at least one symptom either temporarily or permanently.

### DRAWING

FIG. 1 presents three right side diagrammatic views of a human head showing locations of the temporalis, masseter and pterygoid muscles.

### DESCRIPTION

Our invention is based upon the discovery that a therapeutically effective amount of a Clostridial toxin, such as a botulinum toxin (including botulinum toxins types A, B, C₁, D, E, F and G) can be used to assist various dental therapies and procedures. Thus, a botulinum toxin can be used according to a method within the scope of our invention to reduce (including elimination of) a force exerted by a mastication muscle, where such a force impedes healing or interferes with a dental treatment.

Additionally, a botulinum toxin can also be used in a method within the scope of our invention to improve and accelerate re-attachment of a tissue after an oral trauma, infection or treatment (thereby counterbalancing the negative effects of Wolf's Law). Furthermore, a botulinum toxin can be used according to a method within the scope of our invention to accelerate tooth movement in a patient during orthodontic treatment by allowing the dominant vector of force to be derived from the orthodontic appliances. Finally, a botulinum toxin can be used in a method within the scope of our invention to prolong the life of all dental materials and natural tooth in a patient by limiting the excessive and destructive natural biting forces in patients that have compromised tooth strength relative to the force of their bite.

We have determined that a neuromuscular disharmony of the gnathological system impacts the cranium to jaw relationships, mandibular position, the temporomandibular joint position and dental occlusion. The entire gnathalogical system adapts to enable chewing function. Evidence of neuromuscular disharmony of the gnathological system can include: a musculoskeletal occlusion, as evidenced by any of the following: headache, TMJ pain, TMJ noise Jaw clicking), ear congestion, limited opening, vertigo (dizziness), tinnitus (ringing in the ears), dysphagia (difficulty swallowing), loose teeth, clenching, bruxism, facial pain (non-specific), difficulty chewing, tender or sensitive teeth, cervical pain, postural problems, paresthesia of fingertips (tingling), thermal sensitivity (hot and cold), trigeminal neuraligia, Bell's palsy, nervousness and insomnia.

Intra-oral signs of neuromuscular disharmony of the gnathological system can include: crowding lower anteriors, wear of lower anterior teeth, lingual inclination of lower anterior, lingual inclination of upper anterior, bicuspid drop-off, depressed cure of spee, lingually tipped lower posteriors, narrow mandibular arch, high vaulted palate, midline discrepancy, malrelated dental arches, tooth mobility, flared upper anterior teeth, facets, cervical erosion, locked upper buccal cusps, fractured cusps, chipped anterior teeth, loss of molars, open interproximal contacts, unexplained gingival inflammation and hypertrophy, crossbite, anterior open bite, anterior tongue thrust, lateral tongue thrust, and scalloping of lateral border of tongue.

These musculoskeletal occlusion and intra-oral evidence of neuromuscular disharmony of the gnathological system are believed to be due to one or more of the following: (1) a constricted chewing pattern; the front teeth trap movement and development of the jaw. This can cause excessive wear on the front teeth. (2) Occlusal dysfunction; inefficient use of the masticatory muscles creates abnormal wear pattern and chewing function. Premature contact between back teeth when closing create chewing interferences. (3) Parafunction; destructive use of the gnathalogical system for no functional purpose and/or (4) Neurological disorders; destruction of the system for no functional purpose.

These indications of functional occlusal problems can be a result of the gnathological system's attempt to adapt to forces upon it by the mastication muscles. For example, a high vaulted palate can result in particular mastication muscle force vectors being applied to oral structures, which can result in an abnormal mandibular posture and a malocclusions (deep overbite). Thus, functioning components of the gnathalogical system are interrelated and function together, as the teeth, the muscles and joints are connected to the same bone, the mandible. Even though adjustments can be made by equilibration techniques to adjust and make the teeth fit seemingly perfectly together, by ignoring the pathologic joints that are strained and torqued in the glenoid fossa (i.e. by overlooking mastication physiology) future disharmony and accommodated compensations to other bodily structures can result.

In the 19th century surgeon Julius Wolff proposed that mechanical stress was responsible for determining the architecture of bone. Remodeling of bone occurs in response to a physical stress or to a lack of a physical stress upon a bone. Thus, bone is deposited in sites subjected to stress and is resorbed from sites where there is little stress causing the architecture of a bone to be influenced by the mechanical stressed upon the bone as it functions.

One can postulate therefore that a mechanical stress, or equivalently a force applied, can also influence the architecture of all oral tissues during function and healing including bone, muscle, ligaments, tendons. We have discovered that a Clostridial toxin can be used to limit the intensity, duration and frequency of undesired forces during healing following a dental procedure, therapy or treatment.

Thus, a Clostridial toxin can be used: (a) to effect the frequency, duration (stamina) and intensity (force) of a masticatory muscle contraction; (b) facilitate and allow the location of a comfortable neuromuscular bite position to be located and maintained; (c) allow the muscles of mastication to be re-programmed; (d) facilitate masticatory behavioral modification, and; (e) alter (lower) the resting muscle tone of the muscles of mastication

We have discovered that a main cause of dental disease that leads to tooth and surrounding tissue damage is excessive force brought to bear on the dentition by the muscles of mastication. In the normal physiologic rest position there is no contact of the teeth except when eating and even then food should be present between the teeth. The vertical separation between the teeth with the mandible in the neuromuscular relaxed position (NMP) is termed ideal freeway space. As it well known, the phrase “freeway space” means the space between the occluding surfaces of the maxillary and mandibular teeth when the mandible is in physiologic resting position. Freeway space is synonymous with interocclusal clearance, interocclusal distance, interocclusal gap and interocclusal rest space. For normal function and dental health the ideal freeway space should be maintained. Proprioceptive nerves in the periodontal ligaments provide central nervous system input for jaw position. Freeway space is abolished whenever the teeth occlude. When the relaxed jaws close the teeth should comfortably interdigitate without traumatizing each other. However if the teeth do not interdigitate into each other, they meet with excessive forces. When the closed bite is not in the neuromuscular relaxed position, destruction of the teeth and periodonteum occur when the teeth occlude. This force causes loss of tooth material, tooth loss, impedes regeneration of damaged periodonteum, impedes integration of periodontal grafts and implants from being secured in bone and can damage dental hardware.

Orthodontic treatments are hindered by misdirected vertical forces that slow down movement of dentition. Excessive force also produces mal-development of the bite.

The following dental conditions can all be produced by misdirected mastication muscle force: abfraction, gingival recession and bone loss, followed by sensitivity, tooth decay and ultimately loss of teeth. This occurs in mouths where the strength of the enamel and dentine of the teeth cannot withstand the force of the individual's bite.

As disclosed herein, a botulinum toxin can be used to reduce muscle activity by injecting the toxin into the muscles of mastication, which are the temporalis, masseters, and ptyergoid muscles. Typically, from about 50 units to 150 units of a botulinum toxin type A (BOTOX®) can be used per treatment session.

It is important to note that our invention encompasses methods to assist a dental procedure, including a dental therapy and a dental treatment. Our invention is not directed to and excludes simply treating an involuntary patient activity such as bruxism, jaw clenching or tooth clenching. Additionally, our invention is not directed to and excludes simply treating a voluntary patient activity, such as lip biting, jaw clicking or a tooth grinding activity. Our invention is limited to methods whereby a Clostridial toxin, such as a botulinum toxin, is used in conjunction with (typically the Clostridial toxin is administered prior to commencement of the dental procedure) and to assist a dental procedure. A “dental procedure” means an activity carried out by a dentist or by a person with formal training in gnathological (within the chewing apparatus) therapeutic, diagnostic or cosmetic treatment upon humans.

Furthermore, an activity within the scope of a “dental procedure” is only either: (a) an activity in which a natural substance (such as a tooth, bone or tissue) is implanted, transplanted or adhered to or in a substrate in the mouth of a patient, or (b) an activity in which a dental artifact (such as a dental restorative or cosmetic material ) is placed in or on a location within the mouth of a patient.

Bruxism can be defined as the involuntary masticatory muscle activity not used to assist mastication. Thus, treatment of dental conditions such as bruxism or clenching by administration of a botulinum toxin to a patient is excluded from the scope of our invention because such treatments are not within the scope of a dental procedure, as defined above.

We have discovered that a Clostridial toxin can be used to reduce and/or prevent damage to teeth, oral tissues and restoration by de-programming muscles responsible for the relatively excessive functional force. Preferably, the toxin used is a botulinum neurotoxin. The Clostridial neurotoxin improves and accelerates re-attachment of all oral tissues after trauma, infection or treatment

It is known that a botulinum toxin can be used to denervate a muscle. We have found that a botulinum toxin can be used to block neuromuscular activity upon administration to masticatory muscle which is responsible for an unnecessary excessive force. Use of a botulinum toxin according to our invention results in a limited paralysis of the target muscle to thereby alleviate a detrimental force and facilitate unimpeded therapy and healing on all oral tissues including teeth, gums, bone (as per Wolff's Law), ligaments, tendons and muscles.

Thus, a Clostridial neurotoxin can be used to prolong the life of a dental restoration and of weakened teeth by limiting the overloaded natural biting forces in individuals that have compromised tooth strength relative to the force of their bite.

A method according to our invention can be carried out by intramuscular administration (as by injection) of an effective amount of a botulinum toxin to a mastication muscle of the face or mouth of a patient, thereby reducing a detrimental force vector associated with force applied by the targeted muscle. The botulinum toxin can be administered in an amount of between 0.01 units and 500 units (for a botulinum toxin type A, sold as BOTOX®). Such a dose can provide the desired force relief for about three to four months, and administration of the botulinum toxin can thereafter be repeated as often as necessary. Often a de-programming of the muscle eliminates the necessity for repeated doses. By de-programming is it meant that the patient to which the botulinum toxin has been administered learns to use his muscles in a different way. Thus, upon administration of a botulinum toxin, the muscle is weakened and thereafter (until the effect of the administered botulinum toxin wears off) cannot apply the same amount of force (i.e. upon chewing) as it could prior to the botulinum toxin administration. When the muscle is weakened a patient, either consciously or unconsciously, will typically compensate for the loss or reduction of a force vector from that muscle by using the muscle in a different way (i.e. by altering the characteristics, such as direction, longevity, of his biting or chewing profile) or by using adjacent or affiliated muscles in a different manner in conjunction with the weakened muscle. Often such a new behavior can become permanent, as the patient becomes used to it, and his mouth adjusts to it, and the new behavior (the de-programmed muscle activity) is retained even after the muscle weakening effect of the botulinum toxin has worn off. This is a clearly desirable outcome since the prior behavior (amount of force vector applied exerted by the mastication muscle was detrimental) by the muscle was in one or more ways detrimental to an oral tissue, bone, tooth or dental artifact in his mouth. In this way, a patient's muscle can be deprogramming from it's detrimental behavior or effect.

Thus, simultaneous bilateral botulinum toxin injections to facial muscles can be carried out without embarrassment to the appearance and function of the mouth. The direction of muscle fibers and contraction forces are re-aligned and the function of the patient's chewing is not impeded.

We have discovered that by determining (i.e. by quantitating) an excessive force in the mouth and then by reducing such an excessive force in a controlled, measured manner by administration of a botulinum neurotoxins numerous patient benefits can be obtained. These benefits include the following:

1. certain headaches can be relieved. In a Chronic Clenching Syndrome the patient bites and maintains his bite in a fixed position by maintaining contraction of a muscle, such as the temporalis muscle. The chronicly clenched muscle eventually goes into a spasm condition, and pain and headache. Administration of a botulinum toxin according to a method disclosed herein can reduce the muscle spasm (with a concomitant reduction of an excessive bite force), thereby treating the headache.

2. Dental sensitivity can be reduced. Trauma to the periodontal ligament caused by clenching can result in an inflammation of the peridontal ligament and of dental pulp which manifests as a temperature sensitivity. Administration of a botulinum toxin according to a method disclosed herein can reduce the inflammation as well as reducing the clenching force), thereby treating the dental sensitivity. A patient that presents with tooth sensitivity and pain but no discernable tooth pathology can be treated with a botulinum toxin to relax the jaw and open the bite. This enables inflamed ligaments and pulps to return to their normal non-inflamed non-sensitive state.

3. Neck strain can be treated. The head tends to tilts backwards to compensate for a constant or chronic clenching on the front teeth and can tilt forward to compensate for clenching on the back teeth. Both types of head tilt can cause fatigue and cramping in the neck muscles. Thus, the head tilts to adapt for an uncomfortable or mal-aligned bite. When all the back teeth are missing the patient has to move the jaw forward to bite on the front teeth. Thus, the muscles of the back of the neck have to work harder to support a mandible that has to protrude for chewing function. The muscles of the front of the neck work harder to balance the head when the bite is retrusive. Both cause fatigue and cramping in the neck muscles that have to support a head that is not balanced at the top of the spine. Administration of a botulinum toxin according to a method disclosed herein can reduce the clenching (with a concomitant reduction of an excessive clenching force), thereby treating the neck strain. Thus, a patient that presents with a malocclusion and neck pain can have Clostridial; neurotoxin assisted bite correction This can I correct the postural defect and eliminate the neck pain.

4. Certain types of tinnitus can be treated. Clenching of the jaw muscles can cause the tensor tympani and tensor levi palatini muscles within the ear to tense, strain and cause a ringing noise to be perceived by the patient. Administration of a botulinum toxin according to a method disclosed herein can reduce the clenching (with a concomitant reduction of an clenching force), thereby treating the tinnitus.

5. Positioning and maintenance of dental materials and structures can be improved. Administration of a botulinum toxin according to a method disclosed herein can reduce a force which can be applied by the injected muscle. Limiting a muscle biting force in this manner after periodontal surgery, such as after placement of a tooth crown (especially where there is limited crown to bone length) can prevent a muscle applied biting or chewing force from jeopardize the stability of an intra-oral dental artifact and can also prevent tooth loosening. In this manner positioning and maintenance of intra-oral dental materials and structures can be improved.

6. Gum recession can be treated. Gum recession can be caused by chronic application of a lingual and/or facial force vector. Thus, gum and bone are lost in front of the direction of force, especially in when the force is directed towards the lip or cheek side of the tooth. Additionally, torquing the gum and bone away from the periodontal ligament attachments can cause gingival recession and bone loss, followed by sensitivity and decay on these teeth. Administration of a botulinum toxin according to a method disclosed herein can reduce the muscle force thereby treating the gum recession.

7. Tooth and bone loss can be treated. Bone loss associated with either advanced periodontitis or osteoporosis provides less support for the teeth. Regular biting force upon or in the vicinity of such a compromised bone or tooth position can lever the remaining bone away from the toot roots causing accelerated bone loss, and further loosening and loss of teeth. Administration of a botulinum toxin according to a method disclosed herein can reduce the bite force, thereby reducing bone loss and tooth loss.

8. Healing of damaged oral tissues can be facilitated. Limiting a muscle contraction (by administration of a botulinum toxin according to a method disclosed herein) after a trauma to an oral muscle, tendon, ligament or bone can reduce a tensile strain on the damaged tissue, thereby allowing healing of the damaged oral tissue in a reduced time with less pain and minimal internal or external fixation during repair and rehabilitation.

9. Tooth implant retention can be increased and facilitated. When one or more tooth implants are placed application of an muscle force can overload the implant before osseo-integration of the implant has occurred. Thus application of an oral muscle force can cause implant failure due to either fracture or loosening of the implant or an implant component or prevention osseo-integration of the implant with adjacent bone and tissue. Administration of a botulinum toxin according to a method disclosed herein can reduce the muscle force applied, thereby increasing tooth implant retention and reducing a loss of bone material and of bone density by bone adjacent to the implanted tooth.

10. Retention of a patients, natural, re-implanted tooth can be increased and facilitated. A traumatically avulsed tooth can be re-inserted, but such a re-inserted tooth can lose it's vitality and not re-attach to alveolar bone when loaded with relatively excessive functional force. Administration of a botulinum toxin according to a method disclosed herein can reduce a muscle force applied, thereby increasing and facilitating retention of a patients' native, re-implanted tooth.

11. Tooth abfraction can be reduced. It is known that teeth have the ability to flex, usually at the enamel-dentine junction. The crystalline enamel of the flexing tooth can fracture at the point of maximum flexation next to the gum while grinding. The resultant loss of enamel and groove on the sides of the teeth makes them sensitive and more susceptible to decay. Thus, administration of a botulinum toxin according to a method disclosed herein can reduce the an oral muscle force applied, thereby reducing a tooth abfraction.

12. The useful life of a dental restorative material can be increased. Administration of a botulinum toxin according to a method disclosed herein can reduce a force vector applied by a mastication muscle upon an intra-oral dental artifact. In this manner a temporary or a permanent dental restorative material can be protected or sheltered from a destructive force with a resulting longer useful lifespan of the dental restorative material, which dental restorative material can be a dental acrylic, resin, composite, glass ionomer, amalgam, ceramic, porcelain, vitallium, chrome, cobalt, fiber re-enforced post, titanium or stainless steel post, or zirconia dental restorative material.

13. The useful life of a denture, clasp and attachment can be increased. A denture, clasp and attachment can fracture upon continued application of a chewing muscle force, especially when the denture opposes natural teeth. Administration of a botulinum toxin according to a method disclosed herein can reduce an oral muscle force applied, thereby increasing the useful lifespan of a denture, clasp or attachment.

14. Fractures can be reduced or prevented. Administration of a botulinum toxin according to a method disclosed herein can reduce an oral muscle force applied, thereby protecting a natural or an artificial tooth material, including cusps, clasps, attachments, posts and roots from a fracturing with can occur upon excessively forceful chewing function. The result is an increase in the useful lifespan of the tooth or of the dental material.

15. The useful lifespan of a temporary crown or restoration can be increased. It can be important to maintain a vertical dimension during prolonged temporization. Thus, a tooth or implant can be covered with a temporary crown before application of a permanent crown. The temporary crown can be used to open the bite and can be left in place for a prolonged period of times (up to a few years). In a mouth where the occlusion has been destroyed by very aggressive chewing function a temporary crown often do not last, or is prematurely worn down before the therapeutic effects of bite opening can be assessed. A temporary crown can also left in place for a prolonged period while the dentist is waiting for periodontal healing to occur, or to assess how much to open the bite, or in a circumstance when the patient cannot afford a permanent crown. Administration of a botulinum toxin according to a method disclosed herein can reduce an oral muscle force applied, thereby protecting a temporary crown from a muscle force, thereby increasing the lifespan of the temporary crown.

16. An orthodontic treatment time can be shortened by reducing a load on a tooth, especially when there exist a very powerful vertical component of force on tooth and bone. Administration of a botulinum toxin according to a method disclosed herein can reduce an oral muscle force applied, thereby reducing an orthodontic treatment time.

17. Administration of a botulinum toxin according to a method disclosed herein can also reduce a bone loss during an orthodontic procedure which can be caused by traumatizing teeth that interfere with a comfortable bite while the traumatized teeth are being moved and while they are out of occlusion.

18. Development of a deep overbite can be reduced. An excessive vertical pull on the jaws by the jaw closing muscles plays a major part in development of a deep overbite especially during facial development. Thus, administration of a botulinum toxin according to a method disclosed herein can reduce a vertical pull on the jaws by weakening the jaw closing muscles, thereby treating development of an overbite.

19. Tongue thrust can be treated. Anterior, lateral or bilateral tongue thrust can be caused by swallowing while the tongue is positioned between the teeth to form an oral seal while swallowing (instead of behind the upper front teeth). Administration of a botulinum toxin into the geniolossus according to a method disclosed herein can reduce the tongue thrust.

20. Increased mouth opening ability. Limited jaw opening is often caused by muscle spasm and not having opened the mouth wide for a prolonged period of time. This can be due to weakness of the mouth opening muscles or the closing muscles (temporalis, masseter) over-dominating the mouth opening muscles (digastric, platysma, pterygoids). A patient that presents with limited mouth opening ability can have the temporalis and masseters infiltrated with a botulinum toxin. This can permit sufficient opening to allow comfortable wide opening e.g. for eating, dental treatment, shouting, kissing, etc.

21. Jaw muscle spasm caused by Chronic Clenching Syndrome can occur when the muscles are not permitted to relax, even when the patient's mouth is not closed nor chewing. The resting muscle tone does not allow lactic acid clearance. This is common when the jaw does not rest in an ideal neuromuscular position

A patient that presents with muscle cramp and pain can be administered a Clostridial neurotoxins into these muscles enabling them to relax, thereby alleviating the jaw spasm.

22. Torquing the gum and bone away from the periodontal ligament attachments causes gingival recession and bone loss, followed by sensitivity and decay on these teeth. The forces traumatizing the teeth also contribute to sensitivity.

23. Bone loss associated with either advanced periodontitis or osteoporosis provides less support for the teeth. Regular and excessive biting forces in this compromised position will lever the remaining periodonteum away from the roots causing accelerated gum recession, bone loss, loosening and loss of teeth. The roots become exposed to the oral environment. The roots are not covered with protective enamel so they decay more readily

24. Gum and bone are lost in front of the direction of force especially when there is limited root in the alveolar bone. Following periodontal surgical procedures the traumatized tissues need to be protected and not exposed to regular functional forces in the mouth These forces can be altered and reduced with a botulinum neurotoxin and by placing splints and/or changing the occlusion. Resting muscle tone is also reduced.

A periodonticaly compromised patient requiring periodontal surgery can have a botulinum neurotoxin administered into a muscle of mastication prior to surgery to assist with the limitation of forces to the periodonteum. These force modifications are best tolerated by deprogramming the muscles prior to surgery and bite or occlusal altering therapies. This will increase patient comfort and accelerate the healing process.

25. Limiting muscle contraction after trauma to muscles, tendons, ligaments or bone reduces tensile strain on the damaged tissue allowing healing in reduced time with less pain and minimal internal or external fixation during repair and rehabilitation. A patient with damaged, weakened or fractured bones that have muscles attached to them e.g. a broken or severed osteoporotic mandible, will have neurotoxins injected into the masseter and temporalis so that these muscles don't work against the forces holding the damaged bone together

26. When multiple implants or immediate loaded implants are placed—to prevent overloading the implants before and after osseo-integration. Overloading the implants results in implant failure either by fracture or loosening of the implant components or prevention of osseo-integration. A patient that needs teeth placed on implants will require ideal mandibular position and occlusion to limit abnormal force on to the teeth. A botulinum neurotoxin can be administered prior to loading these implants with tooth prostheses.

27. Re-implanting teeth that have been traumatically avulsed. Often this is accompanied with damage to the surrounding PDL and bone. The loosened teeth will lose their vitality and not re-attach to alveolar bone when loaded with relatively excessive and mis-directed functional force The teeth may also be splinted, and a bite guard may be placed in the mouth. Restricted force is required until the ligaments re-attach. A patient that presents with numerous loosened and avulsed teeth can benefit from a botulinum neurotoxin administration to a mastication muscles to protect the tissues as they heal.

28. The muscles of the stomagnathic system should be in an ideal state to position the mandible in an ideal neuromuscular position. If the position of the jaw is not in this ideal position as the teeth make contact they will skid, slide and grid along each other until the teeth fit into each other. This leads to worn down, abfractured teeth; teeth have the ability to flex, usually at the enamel-dentine junction. Abfraction is when the crystalline enamel of the flexing tooth fractures at the point of maximum flexation next to the gum while grinding. The resultant loss of enamel and groove on the sides of the teeth makes them sensitive and more susceptible to decay), decayed and broken teeth and damage to all temporary and permanent dental restorative materials, tooth repairs and replacements including all restorations, dentures, crowns, bridges, implants, transplants and newly generated teeth.

29. Maintaining vertical dimension during prolonged temporization. Teeth are covered with temporary crowns before being crowned. These temporary crowns are used to open the bite are often are left for prolonged period of times up to a few years. In a mouth where the occlusion has been destroyed by very aggressive chewing function these temporaries often do not last, or are prematurely worn down before the therapeutic effects of bite opening can be assessed. Temporary crowns are also left for prolonged periods when the dentist is waiting for periodontal healing, assessing how much to open the bite or when the patient cannot afford permanent crowns.

Thus, it is clear that a predominate factor in many dental pathologies is the presence of an excessive or destructive forces transferred through the teeth to the periodontium and TMJ, and we have discovered that administration of a botulinum toxin according to a method disclosed herein can reduce such an excessive or destructive force to thereby treat many disparate dental pathologies.

Furthermore, every restorative, cosmetic, periodontal or orthodontic treatment has a risk of failure resulting from the potentially destructive parafunctive forces of occlusion. We have discovered that administration of a botulinum toxin according to a method disclosed herein can reduce such a force to thereby facilitate the success of many disparate restorative, cosmetic, periodontal and orthodontic treatments. Essentially, we have discovered that by decreasing a force supplied by an oral musculature we can decrease the possibility of a failure or suboptimal of a therapeutic dental procedure.

As set forth, we have discovered that an effective way to prevent damage to a patient's gnathalogical system including dental hard tissue and restorations is to de-program the muscles and reposition the teeth responsible for the excessive adaptational functional force.

According to our invention, a botulinum neurotoxin capable is administered to a mastication muscle responsible for a force exerted upon an oral location. The resulting limited toxin induced paralysis of the muscle can reduce the detrimental force, thereby allowing for muscular de-programming and aids unimpeded therapy and healing on all tissues including teeth, gums, bone, ligaments, tendons and muscles. Use of our method improves and accelerates re-attachment of oral tissue after trauma, infection or a dental treatment.

Our method can prolongs the life of all dental restorations and weakened teeth in all mammals by allowing the dentist to make the necessary compensatory and adaptive changes be easily tolerated by limiting the overloaded natural biting forces in individuals that have compromised occlusion and tooth strength relative to the force of their bite.

A method within the scope of our invention can be carried out by administering by intramuscular injection an effective amount of a botulinum toxin to a muscle of the face or mouth of a patient, thereby relieving the specified conditions within one to seven days, wherein the condition is associated with a muscle contraction. The botulinum toxin is administered in an amount of between 0.01 units and 500 units. The dose is effective from three to four months and can be repeated as often as necessary. Often the de-programming of the muscle eliminates the necessity for repeated doses.

Simultaneous bilateral a botulinum toxin injections to facial muscles can be administered without embarrassment to the appearance and function of the mouth. The direction of muscle fibers and contraction forces are re-aligned and the function of the patient's chewing is not impeded. Chronic clenching can only be achieved by conscious effort.

It can be difficult and uncomfortable for the patient to accommodate to a change in occlusion or posture of the mandible due to a dental treatment. Such a change can be more easily tolerated by deprogramming the muscles of the patient that contribute to such discomfort. The muscles of mastication are the major cause of such discomfort and reprogramming (easing the contraction and tension) of these muscles and repositioning the mandible will allow structural change is a solution to this problem. Thus, we have determined that a botulinum neurotoxin can be used by administration to a muscle of the gnathological system to reduce the pain and discomfort.

A botulinum toxin can be used in conjunction with a dental treatment which includes use of a fixed and removable dental appliance or prosthetic therapy, such as a habit appliances, a fixed or removable tooth moving appliances, closing open bites and gaps, correcting cross bites, arch development, functional oro-facial orthopedics, finishing appliances, mouthguards, all protective and re-posturing occlusal splints, splints to enhance restorative dentistry, an implant, a crown, a bridge that change the occlusion in any way, a denture that change the patients mandibular position both vertically and horizontally. (This includes specific infiltration of the obicularis oris and buccinator when building up the flanges to support collapsed cheeks and lips,) an intra and extra-oral anti-snoring device, a sleep apnoea device, an intra-oral and extra-oral bone fixation device, a fixed or removable prosthesis, and a bone augmentation (as may be used due to natural bone insufficiency or following natural recession of the alveolar bone, periodontal disease, infection, cancer or any degenerative disease).

Muscle function and status during function and at physiologic rest can be measured by surface EMG recordings. In this manner one can measure mandibular moment and distinguish abnormal mandibular opening patterns from normal patterns. Taking a bite registration is a common dental procedure used to establish a mandible to maxillary relationship. Upon determination of this relationship it can be used for e.g. crown restoration and denture fabrication.

A bite registration can also be established by using the existing present supportive teeth with the accommodative wear patterns as landmarks. Establishing an optimal mandibular position when the mastication muscles are at a relaxed and unstrained position can be determined to find an optimal normalized joint position. If a bite registration is taken in an accommodated or manipulated position one cannot easily determine an optimal mandible to cranium relationship, optimal tooth morphology or dental architecture and the dentist is thereby prevented from helping the patient to obtain an optimal and stable neuromuscular occlusion. Additionally, a bite registration which is not taken at an optimal mandibular position reduces the possibility of the dentist being able to obtain for the patient a muscular balance of the head, the mandible and neck.

Thus, determining for a patient and then establishing for that patient an optimal neuromuscular bite position can be important for achieving optimal dental function, stability and harmony of the stomatognathic system. Specifically, determining and then establishing an optimal neuromuscular bite position for a patient permits a dentist to: develop and establish optimal dental aesthetics as far as tooth shape, contour, anatomy and morphology in both the anterior and posterior regions; establish optimal facial cosmetics due to a more harmonious muscular balance when an optimal physiologic mandibular position is found as well as the lower one third of the face which is often deficient vertically and aesthetically; address the numerous musculoskeletal occlusal signs and symptoms which often go undetected such as: headaches, ear congestion feelings, ringing in the ears, pressure behind the eyes, teeth sensitivities, TMJ noise, masticatory muscle tenderness, neck and shoulder pain.

The optimal neuromuscular position (at which an accurate bite registration can be made) of the mandible exists for mandibular vertically anterior, -posterior and lateral positions when the head is in an upright postural position and the involved muscles, particularly the elevator and depressor muscles, are in equilibrium in tonic contraction. It is that position in space where minimal expenditure of muscle energy is needed along an isotonic path of mandibular closure that begins from the rest position of the mandible. This means that the extensor and depressor muscles that move the mandible are postured at a position that exert minimal electrical activity during resting modes.

The optimal neuromuscular position can be measured using surface electromyography (sEMG) or by use of mandibular tracking instrumentation, as a means to visually verify positional changes from a habitual accommodative rest position to a physiologic rest position usually after muscle stimulation via TENS (transcutaneous electro-neuro stimulation).

An accurate bite registration can be obtained after understanding that forces exerted by the masticatory muscles. There are five mastication muscles. The temporal, masseter, medial pterygoid, upper lateral pterygoid and lower lateral pterygoid muscles. These muscles are innervated by the fifth cranial nerve and they are directly responsible for application of stress to oral tissues.

The amount of a Clostridial toxin selected for local administration to a target tissue according to the present disclosed invention can be varied based upon criteria such as the severity of the ailment being treated, the extent of muscle tissue to be treated, solubility characteristics of the neurotoxin toxin chosen as well as the age, sex, weight and health of the patient. For example, the extent of the area of muscle tissue influenced is believed to be proportional to the volume of neurotoxin injected, while the quantity of a muscle weakening effect is, for most dose ranges, believed to be proportional to the concentration of a Clostridial toxin administered. Methods for determining the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill).

### EXAMPLES

The following examples illustrate aspects and embodiments of the present invention. In each of the examples below any of the botulinum neurotoxin serotypes can be used in suitable amounts, alone or in combination.

### Example 1

### Use of a Botulinum Toxin to Establish an Optimal Oral Neuromuscular Position

This example demonstrates that a botulinum toxin can be used to assist establishment of an optimal bite position. A botulinum toxin type A purified neurotoxin complex can be used (BOTOX). Two patients were studied.

At a baseline visit (T₀) any excessive wear and breakdown of teeth is noted in each patient. Dental x-rays are taken with documentation of widened periodontal ligament space, bone loss, and loss of lamina dura. The patient's gingival recession and history of fractured teeth is taken. The jaw position was visualized using a computerized mandibular scanning device (available from Myotronics-Noromed, Tukwila, Wash.).

Computerized Mandibular Scanning is an assessment of mandibular function using biomedical instrumentation, which measures the rotational movement in the frontal and sagittal planes thus confirming a neuromuscular dysfunction. The computerized mandibular scanner measures jaw movement (both qualitatively and quantitatively in several dimensions) to within 0.1 millimeters of accuracy. With a magnetic tracking device and sensor array, it projects the data on a calibrated computer monitor.

The Computerized Mandibular Scanning measurement of jaw movement is more accurate than the eye, making it possible to document characteristics of mandibular motion considered significant to evaluate jaw function. It also identifies the amount of free space, the swallowing pattern, the quality of the occlusion, substantiates the presence of disc derangements and their prognosis for reduction. It is a multi-dimensional assessment of torquing movements used to differentiate between contributing factors of a pathologic position to a non-pathologic position on opening and closing of the mandible.

Graphic recording of opening and closing paths of jaw movements from the side and front views can be analyzed to assess abnormal mandibular paths of movement. The speed at which the jaw can open and close can be simultaneously recorded. Graphical recordings were made using computerized mandibular scanning of sagittal/frontal views of jaw movement to thereby record a range of motion.

Surface electromyography equipment (Myotronics-Noromed) was also used to measure muscular activity. Surface electromyography can be used to specifically delineate and define muscle activity. Surface electrodes were be placed over the muscles which in turn send impulses to the recording instrument. The surface electromyography equipment illustrated used eight channels to monitor the right and left posterior temporalis muscles, right and left anterior temporalis muscles, right and left masseters, and right and left anterior digastric muscles. A strained jaw position can effect muscle activity. The objective is to determine the optimal resting jaw position at physiologic rest that harmonizes with resting EMG levels. sEMG recordings were made of the jaw at rest (not in the neuromuscular related position for that patient).

Clinical measurement of maximum voluntary bite force was carried out using calibrated Flexiforce sensor strips (Tekscan, Inc., Boston, Mass.) attached to an Ohmmeter (Extech Products Quality Instruments, Inc., Tampa, Fla.). The resistance measured in Ohms using the sensor strips was converted to pounds of biting force. The FlexiForce sensors were used to measure force between two bite surfaces. Each sensor was calibrated so that the reading on the multimeter in Ohms of resistance was converted into pounds of force (weight).

The sensors were calibrated with a calibration device that could focus force on to the active sensing area. Two wooden platforms 10″×12″×2″× were attached to a wooden rectangular block 10″×6″×4″. One platform was fixed to the wooden block to provide a stable base. The other platform was attached with 2 brass hinges to the block to act as a loading platform. A brass doorstop was screwed under this top platform close to the edge away from the hinged side. The rubber protector was removed from the stop. The end of the doorstop had a flat brass tip that fitted perfectly into the active sensing area. The top of the hinged platform was loaded with weights in 10 pound increments while the resistance in Ohms was read off the multimeter (Extech multimeter, available from Radioshack). The platform was loaded up to 200 lbs.

The weight of the upper platform plus doorstop was three pounds. For this reason seven pounds were added to read the resistance for ten pounds. Thereafter weights were added in ten pound increments and the resistance at each weight was recorded, as shown by Table 1.

TABLE 1
Ohm Meter Calibration (conversion of
ohms readout to pounds of weight)

| Weight (lbs) | Resistance (ohms) |
|---|---|
| 10 | 470 |
| 20 | 260 |
| 30 | 185 |
| 40 | 145 |
| 50 | 127 |
| 60 | 112 |
| 70 | 103 |
| 80 | 97 |
| 90 | 93 |
| 100 | 91 |
| 110 | 89 |
| 120 | 88 |
| 130 | 87.5 |
| 140 | 87 |
| 150 | 86.8 |
| 160 | 86.5 |

A temporary crown matrix button (available from Advantage Dental Products), was used to make a flattened biting surface to reduce shearing forces that could damage the teeth and the sensors. The heat softened material was finger molded around the tooth to be used when measuring the force of the bite. The material was extended to the teeth on either side for stops. The opposing upper and lower right first molars were selected. The surface of the button was flattened and marked with a black marker pen so that the exact site where the sensor was placed could be used for each measurement.

Interventional Visit (T₀)

Sterile procedure was used. Botulinum toxin type A (BOTOX) 100 unit vial was diluted (reconstituted) with 2 cc's normal saline and drawn into a 27-30 gauge 27-30, 1½ to ½ needle, tuberculin syringe. The muscles of mastication were injected, the dose depended on baseline muscle size, i.e.: range dependent on body build and hypertrophy of muscles.

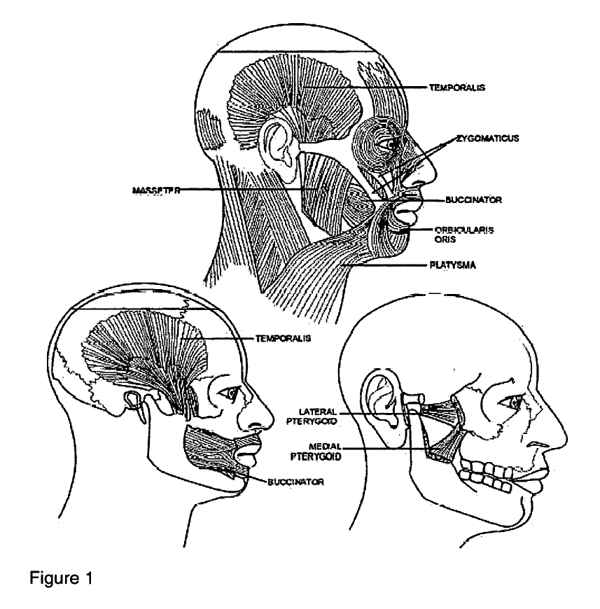

The muscle doses used were 10-50 units into the temporalis muscle and 10-50 units into the masseter muscles of each patient. The masseter (see FIG. 1) was injected transcutaneously through the cheeks into the masseter. The temporalis (see FIG. 1) was injected through the forehead and scalp above the ears. into the temporalis. However these muscles can also be injected through the mouth trans-mucosaly.

Follow UP Visits:

Each patient returned for weekly visits for the following four months. Symptoms related to clenching were documented as improved, unchanged, or worse. The position of the head of condyle was located at rest and compared to its position prior to botulinum neurotoxin treatment using Computerized Mandibular Scanning.

Each patients was instructed to clench with maximum force on command for seconds on to Flexiforce sensors. The time that this maximum bite force could be maintained before it started to taper off was measured. Maximum bite force readings were averaged out.

The main outcome measure that was assessed was the reduction in the bite force and the positioning of the head of the condyle. This was assessed for duration of effect of the botulinum neurotoxin. It returned to baseline force and position at 3-4 months post initial treatment. Serial measurements of clinical force were tabulated. Symptomatic improvement and side effect profiles were documented. EMG data was used to verify resting muscle activity. A calm rested mandibular position was also verified via computer mandibular scanning.

No side effects were noted. In particular normal chewing was not affected.

The total dose of Botulinum toxin type A injected once at week 0 (T₀) was:

Patient H:

Masseters: Left 20 units+10 units in 2 cc dilution

Right Left 20 units+10 units in 2 cc dilution

Temporalis: Left 20 units+10 units in 2 cc dilution

Right 20 units+10 units in 2 cc dilution

Total dose in patient H at this T₀ (week 0) treatment session: 120 units.

Patient D:

Masseters: Left 20 units in 2 cc dilution

Right 20 units in 2 cc dilution

Temporalis: Left 20 units in 2 cc dilution

Right 20 units in 2 cc dilution

Total dose in patient D at this T₀ (week 0) treatment session: 80 units.

TABLE 2
Bite force and time to muscle fatigue after bilateral botulinum toxin
injection into masseter and temporalis muscles of two patients

| | |
|---|---|
| Patient D | Patient H |
| (ectomorph female) | (mesomorph male) |

| | Force | Fatigue time | Force | Fatigue time |
|---|---|---|---|---|
| Week | (lbs) | (seconds) | (lbs) | (seconds) |
| 1 | 57 | 6 | 167 | 11 |
| 2 | 59 | 6 | 165 | 12 |
| 3 | 51 | 4 | 149 | 8 |
| 4 | 48 | 2 | 147 | 7 |
| 5 | 49 | 1 | 148 | 5 |
| 6 | 49 | 1 | 145 | 4 |
| 7 | 46 | 1 | 146 | 4 |
| 8 | 47 | 2 | 147 | 5 |
| 9 | 48 | 2 | 139 | 7 |
| 10 | 49 | 1 | 144 | 6 |
| 11 | 45 | 1 | 146 | 5 |
| 12 | 46 | 1 | 150 | 8 |
| 13 | 49 | 2 | 159 | 8 |
| 14 | 52 | 3 | 165 | 7 |
| 15 | 53 | 4 | 166 | 8 |
| 16 | 50 | 4 | 170 | 5 |
| 17 | 55 | 4 | 168 | 6 |
| 18 | 56 | 4 | 166 | 7 |

It was noted that after injection the temporalis and masseter muscles were tender and that once the toxin had taken effect there was rapid fatigue when chewing. This lasted from week 3 until week 10 on both patients.

Both patients condyles were in the neuromuscular relaxed position at rest. Both patients were aware of a decreased tension and tenderness around the temporalis and masseter muscles (after about week 2). Neither of the patients were aware of any limitation to chewing regular foods. Thus, as set forth by this example, an optimal oral neuromuscular position can be obtained by first determining the bite position of a patient, followed by administering a botulinum toxin to a mastication muscle of the patient where the mastication muscle exerts a force which prevents attainment of an optimal oral neuromuscular position. One waits a period of time sufficient for the botulinum toxin to weaken the mastication muscle, thereby obtaining an optimal oral neuromuscular position, as shown by a second bite position determination, as for example by the methodologies set forth above.

### Example 2

### Use of a Botulinum Toxin to Facilitate a Tooth Implant

A female patient age 34 presents with her four upper incisor teeth having been traumatically avulsed in a racquetball accident. All four teeth are dangling in the palate. They are held only by limited gingival attachments. The patient is seen by her dentist within an hour of the accident. Her primary dentist re-positions the teeth within their sockets and splints them together. At the time of the dental procedure, the patient's masseter and temporalis muscles are injected on both sides of the mouth with a total of eighty units of a botulinum toxin type A. The resulting weakening of the closing masticatory muscles can reduce a force that is applied to the location of the re-implanted teeth and adjacent tissues when the patient chews, swallows, brings her teeth together or smiles. The mandible can remain in a passive posture during rest and freeway space can be is maintained. With reduced masseter and temporalis muscle forces being applied to the implanted teeth they can rapidly re-attach to the traumatized periodontal ligaments, adjacent bone and tissue. The teeth can also all revascularize and remain vital. The mandible can remain passive preventing unnecessary rocking forces being transmitted through the teeth to the attachment tissue. Inflammation can be reduced during the healing process. Alternately, the patient can be injected with 4000 units of a botulinum toxin type B.

This example shows that retention of a re-implanted tooth as well as the re-implanted tooth's vitality can be increased and facilitated by use of a botulinum toxin according to our invention. Use of the botulinum toxin in the method disclosed above can prevent application by the patient's mastication muscles of an excessive or mal-aligned force which can overload the periodonteum before re-attachment of the teeth has occurred. Thus, use of a botulinum toxin has prevented application of an excessive oral muscle force which can cause re-attachment failure and loss of vitality due to either compression or fracture of surrounding periodonteum or loosening of the teeth. Furthermore, the teeth are less prone to external resorbtion because they remain vital.

It can be observed that the implanted tooth adheres to the implant site (the substrate), as evidenced by the stability and the vitality of the implanted tooth, in a time period which is at least 20% shorter as compared to the time period required for substrate adhesion by a tooth implanted in an identical or in a similar tooth implant procedure where a botulinum toxin is not used.

### Example 3

### Use of a Botulinum Toxin to Facilitate a Tissue Transplant

A male patient 42 presents with several areas of advanced gum recession due a previous tobacco smoking habit and due to the lack of attached gingival tissue. The patients mouth has been well cleaned regularly by the patient and the patient has good oral hygiene. Seven days prior to a gum tissue grafting procedure, the patient's masseter and temporalis muscles are injected on the side of the tissue transplant with a total of one hundred units of a botulinum toxin type A. The resulting de-programming and weakening of the muscles of mastication can reduce the forces that are applied to the location of the prospective gum tissue transplant and to adjacent tissues when the patient chews, swallows, brings his teeth together or smiles. Additionally, freeway space can be maintained because the jaw closing muscles are no longer as dominant over the opening muscles. A dental gum tissue auto graft procedure is carried out seven days after the botulinum toxin administration. For the transplant, gingival tissue can be harvested from the roof of the patients mouth. Small sections can be grafted onto prepared receptor sites where the gums have receded. With reduced masseter and temporalis muscle forces being applied via the teeth to the new gum tissue grafts, there can be a rapid adherence, re-vascularization and integration of the transplanted tissue to adjacent tissue. Inflammation can also be reduced during the healing process. Alternately, the patient can be injected with 5000 units of a botulinum toxin type B.

It can be observed that the transplanted gum tissue adheres to the graft site (the substrate), as evidenced by vascularization (good red color) and innervation (pain sensitivity) of the transplanted gum tissue, in a time period which is at least 20% shorter as compared to the time period required for substrate adhesion by a gum tissue transplanted in an identical or in a similar gum tissue transplant procedure where a botulinum toxin is not used.

### Example 4

### Use of a Botulinum Toxin to Reduce Dental Sensitivity

A patient presents with sensitive teeth and gums. He has experienced trauma and inflammation to the pulps and periodontal ligaments caused by clenching. Inflammation to the peridontal ligament and the pulps of several teeth on one side of his mouth has manifested as temperature and pressure sensitivity. The patient's masseter and temporalis muscles can be injected with a total of 120 units of a botulinum toxin type A. Alternately, the patient can be injected with 6000 units of a botulinum toxin type B. Within about one to seven days the resulting reduced contraction and weakening of the mastication muscle can reduce the force that is applied to the location of the sensitive teeth and gums, resulting in a reduced tooth and gum sensitivity. The patient subsequently can also undergo occlusal adjustment therapy. The forces on the teeth can thereby become evenly distributed. Subsequently, the patient can easily tolerate the new mandibular position and the sensitivity and pain do not reoccur.

### Example 5

### Use of a Botulinum Toxin to Reduce Neck Strain

A patient presents with a strained neck. An intra-oral exam can reveal that he has an anterior open bite and missing second bicuspids or molars on the upper jaw (maxilla). His head tilts forwards to occlude and to compensate for the missing teeth. He has a chronic clenching habit and has to position the mandible in a neuromuscular non-rest position. The patent's teeth can come together thousands of times each day. He postures his head forward to compensate for the abnormal posturing of the mandible. The head tilt can cause fatigue and cramping in his neck muscles. The patient's masseter and temporalis muscles are injected with a total of 120 units of a botulinum toxin type A. Within about one to seven days the resulting weakening of the masticatory muscles reduces the force, duration and frequency of his teeth clenching, resulting in less neck strain. Alternately, the patient can be injected with 6000 units of a botulinum toxin type B with the same result.

### Example 6

### Use of a Botulinum Toxin to Treat Tinnitus

A patient presents with tinnitus caused by constant clenching of the jaw muscles The temporal and lateral pterygoid muscles were found to be very tender to palpation. He is referred by the ear, nose and throat specialist because nothing abnormal was found in the ears. The tensor tympani and tensor levi palatini are tensed when ever the jaw-closing muscles are tensed, i.e., when ever the jaw is clenched. The tensed muscles and mal-positioned mandible applies pressure to the ear causing a ringing noise to be perceived by the patient. The patient's masseter and temporalis muscles are injected with a total of 80 units of a botulinum toxin type A. Within about one to seven days the resulting weakening of the mastication muscle reduces the force of her teeth clenching, resulting in resolution of her tinnitus problem. Alternately, the patient can be injected with 4000 units of a botulinum toxin type B with the same result.

### Example 7

### Use of a Botulinum Toxin to Increase Lifespan of a Dental Artifact

A patient presents with several crowns and veneers that have failed due to abnormal forces placed upon them. The dentist diagnoses that the vectors of force placed upon them are beyond the tolerance of the ceramic material. The patient's masseter and temporalis muscles are injected with a total of 80 units of a botulinum toxin type A. Within about one to seven days the resulting weakening of the mastication muscle reduces the forces applied by these muscles. Alternately, the patient can be injected with 4000 units of a botulinum toxin type B. The patients jaw is de-programmed and the teeth are restored after a new bite position has been established. The patient can easily tolerate the new position, which can limit the applied force on the dental restorations. He can maintain freeway space and the jaw can rest in a comfortable position. Administration of the botulinum toxin can thereby prevent the muscle applied biting or chewing force from jeopardizing the stability of the intra-oral dental artifacts and can also reduce wear upon the dental artifacts. In this manner both artifact location placement and life span can be improved.

Thus, the useful life of all dental restorative materials can be increased. As set forth, administration of a botulinum toxin according to a method disclosed herein can reduce a force vector applied by a mastication muscle upon an intra-oral dental artifact. In this manner a temporary or a permanent dental and periodontal restorative material can be protected or sheltered from a destructive force with a resulting longer useful lifespan of the dental restorative material (dental artifacts). Such dental and periodontal restorative materials include but are not limited to dental acrylic, resin, composite, glass ionomer, amalgam, ceramic, porcelain, vitallium, chrome, cobalt, fiber re-enforced post, titanium or stainless steel post, or zirconia dental restorative material. Additionally, the useful life of an implant, denture, clasp and attachment can be increased. An implant, denture, clasp and attachment can fracture upon continued application of a chewing muscle force, especially when the denture opposes natural teeth. Administration of a botulinum toxin according to a method disclosed herein can reduce an oral muscle force applied, thereby increasing the useful lifespan of a denture, clasp or attachment.

### Example 8

### Use of a Botulinum Toxin to Treat Gum Recession

A patient presents with periodontal recession. This can be caused by chronic application of a force vector by his mastication muscles via his teeth to the surrounding periodonteum due to a chronic clenching habit. Gum and bone has been lost in front of the direction of force, especially in when the force is directed away from the long axis of the teeth. Additionally, torquing the gum and bone away from the periodontal ligament attachments has caused gingival recession and bone loss, followed by sensitivity and decay on these teeth. The patient's masseter and temporalis muscles are injected with a total of 100 units of a botulinum toxin type A. Within about one to seven days there has occurred a weakening of the mastication muscles and a concomitant reduction of the forces applied by these muscles. Subsequently, the rate of both his gum recession and bone loss has been reduced. Alternately, the patient can be injected with 5000 units of a botulinum toxin type B.

### Example 9

### Use of a Botulinum Toxin to Treat Bone Loss

A patient presents with tooth and bone loss. Her bone loss is associated with either advanced periodontitis or osteoporosis and provides less support for the teeth. Despite the bone loss, bite force studies show no reduction in her biting force. A regular biting force upon or in the vicinity of such a compromised bone or tooth position has levered the remaining bone away from the tooth roots causing accelerated bone loss, and further loosening and loss of teeth. Dental x-rays can show a funneled widening of the periodontal ligament spaces and loss of lamina dura around the teeth. The patient's masseter and temporalis muscles are injected with a total of 80 units of a botulinum toxin type A. Within about one to seven days there has occurred a weakening of the mastication muscles and a concomitant reduction of the forces applied by these muscles. Subsequently, the rate of both her bone loss has been reduced. Alternately, the patient can be injected with 4000 units of a botulinum toxin type B.

### Example 10

### Use of a Botulinum Toxin to Assist Healing of Oral Tissues

A patient presents with a fracture at the angle of the mandible following a punch to the jaw. In addition there is traumatized masseter muscle, tendon and TMJ ligaments. The patient's masseter and temporalis muscles can be injected with a total of 80 units of a botulinum toxin type A at the time of diagnosis. The jaw can be immobilized the next day with limited fixation. Within about one to seven days there has occurred a weakening of the mastication muscles and a concomitant reduction of the forces applied by these muscles. In addition the muscles remain more passive than normal at rest. This can result in a reduction of the tensile strain placed upon the damaged tissue, thereby preventing separation of the fracture and permitting healing of the damaged oral tissue in a reduced time with less pain and with minimal internal or external fixation during repair and rehabilitation. Alternately, the patient can be injected with 4000 units of a botulinum toxin type B with the same result.

### Example 11

### Use of a Botulinum Toxin to Facilitate a Tooth Re-insertion

A female patient age 43 has elected to replace her partial denture with six implants. She requests that she have the abutrments and crowns attached to the implants within a week of having them placed into her maxilla. Seven days prior to the implant procedure, the patient's masseter and temporalis muscles are injected on the side of the implant with a total of eighty units of a botulinum toxin type A. The resulting weakening of the mastication muscle reduces the force that is applied to the location of the prospective implants and adjacent tissues when the patient chews, swallows, brings her teeth together or smiles. Crown seating on the implants is successfully carried out. With reduced masseter and temporalis muscle forces being applied to the new implants and teeth, the implant rapidly adheres to adjacent bone and tissue and inflammation is reduced during the healing process. Alternately, the patient can be injected with 4000 units of a botulinum toxin type B. Thus, retention and success of a patients implanted teeth has been increased and facilitated. The implants can successfully integrated with alveolar bone because it is now loaded with a relatively reduced functional force from her mastication muscles.

### Example 12

### Use of a Botulinum Toxin to Reduce Tooth Abfraction

A female patient age 49 presents with generalized tooth abfraction. Her teeth have the ability to flex at the enamel-dentine junction. The crystalline enamel of the flexing tooth has fractured at the point of maximum flexation next to the gum while grinding. The resultant loss of enamel and groove on the sides of the teeth makes them sensitive and more susceptible to decay. Class Five restorations and veneers have failed because the habit had not been eliminated. The patient's masseter and temporalis muscles are injected on the side of the implant with a total of eighty units of a botulinum toxin type A. The resulting weakening of the mastication muscle and reduced tone reduces the force that is applied reduces her subsequent tooth abfraction. Her teeth are restored successfully and the restorations do not separate from the teeth. In addition her abfraction does not recur. Alternately, the patient can be injected with 4000 units of a botulinum toxin type B.

### Example 13

Use of a Botulinum Toxin to Reduce Time Period for an Orthodontic Treatment

A male patient age 23 with a deep bite and oversized masseters presents with a need for braces to treat his prominent buck teeth. His strong bite can prevent the orthodontic arch-wires from effectively moving his teeth. Braces are applied in the normal manner. Concurrently, his orthodontic treatment time is shortened by reducing the load on the teeth, notably a strong vertical component of force on tooth and bone applied by his mastication muscles. This is accomplished by injecting the patient's masseter and temporalis muscles on both sides with a total of 120 units of a botulinum toxin type A one week prior to fitting the bands, brackets and archwires. The resulting de-programming and weakening of the masticatory muscles reduces the force that is applied and thereby reduces a detrimental force vector, hence permitting the correctional force applied by the braces to be exerted unimpeded. His braces can therefore be removed earlier than would otherwise have been be possible. Alternately, the patient can be injected with 6000 units of a botulinum toxin type B.

### Example 14

### Use of a Botulinum Toxin to Treat Snoring

A healthy male patient of 49 has a snoring problem. He requests a snoring prevention device from his dentist. The appliance can successfully stop his snoring. However after 3 nights he can complain of discomfort in and around his TMJ (temporo mandibular joint). He can be unable to tolerate the appliance in his mouth because it re-positions the jaw. He is injected with 120 units of botulinum toxin type A into the masseter and temporalis muscles on both sides of the face. The muscles de-program and allow the patient to wear the device without discomfort. Alternately, the patient can be injected with 6000 units of a botulinum toxin type B.

### Example 15

### Use of a Botulinum Toxin to treat a Locked Occlusion

A 28 year old female patient presents with worn down and fractured cuspid and bicuspids on the right side of her mouth. The teeth have been repaired before but the restorations lasted only two years. Her muscles are in spasm because the mandible cannot close in an ideal position. The dentist finds that her jaw cannot function ideally because she has a locked occlusion. The teeth and restorations have fractured as the mandible attempts to adapt the teeth so that they don't interfere with chewing. He recommends orthodontics followed with crowns on the right side of her mouth. The patient says she cannot afford this treatment but wants some immediate relief for the muscles in spasm. She is given a discluding device which gives some relief at night. She cannot wear it during the day because she is in sales and has to speak all day long. The dentist injects each temporalis and masseter with 20 units of botulinum toxin type A, giving her a total of 80 units. The patient can have complete relief after 6 days. Alternatively the patient could be injected with 4000 units of botulinum toxin Type B.

### Example 16

### Use of a Botulinum Toxin to Treat a Malocclusion

An eleven year old girl presents at the orthodontist with a retrusive mandible and a deep bite. She resembles her mother who has a very deep bite and large hypertrophied masseter muscles. Wrist x-rays reveal that she is not yet finished growing. The orthodontist begins treatment for her malocclusion. The patient is given 80 units of botulinum toxin Type A every 4 months until treatment and growth is complete. The pull of the masseter on the developing mandible is restricted allowing growth without impedance. The mandible develops ideally overcoming genetic influences. The patient can now have an ideal facial profile.

### Example 17

### Use of a Botulinum Toxin to Facilitate Denture Placement and Use

A sixty three year old female requires a new full denture. Her ridges have completely resorbed and her bite has collapsed. In addition she has no lip support. Her face has completely fallen in. The dentist constructs a denture that opens her bite by more than ten millimeters so that her face has a normal vertical height. He also makes the flanges under the lip very thick so that the lip has support. She initially cannot tolerate the forced opening of the bite. He injects her masseter and temporalis muscles with 120 units of botulinum toxin Type A. Within 3 days she can tolerate the rapid and extreme opening of her bite. However she is uncomfortable under her lips due to the thick flange pushing against them. The dentist injects the obicularis oris with 10 units of Botulinum toxin type A. Within 7 days the thick flange under her lip no longer bothers her.

The invention disclosed herein has many advantages including the following:

1. reduce and eliminate relative force imbalances in the mouths of a patient that are destructive, impede healing and interfere with dental treatments;

2. improve and accelerate re-attachment of an oral tissue after trauma, infection or treatment (counterbalancing the negative effects of Wolf's Law);

3. accelerate tooth movement in a patient during orthodontic treatment by allowing the dominant vector of force to be derived from the orthodontic appliances;

4. prolong the life of all dental materials and natural tooth in a patient by limiting the excessive and destructive natural biting forces in individuals that have compromised tooth strength relative to the force of their bite, and;

5. permits easy adaptation to change of shape, form, dimension or position of any or all of the components of the gnathological system by deprogramming the muscles allowing functional behavioral modification.

Although the present invention has been described in detail with regard to certain preferred methods, other embodiments, versions, and modifications within the scope of the present invention are possible. For example, a variety of Clostridial neurotoxins can be effectively used in the methods of the present invention. Additionally, the present invention includes local administration methods to assist a dental procedure wherein two or more neurotoxins, such as two or more botulinum toxins, are administered concurrently or consecutively. For example, botulinum toxin type A can be administered until a loss of clinical response or neutralizing antibodies develop, followed by administration of botulinum toxin type B. Alternately, a combination of any two or more of the botulinum serotypes A-G can be locally administered to control the onset and duration of the desired therapeutic result. Furthermore, non-neurotoxin compounds can be administered prior to, concurrently with or subsequent to administration of the neurotoxin to proved adjunct effect such as enhanced or a more rapid onset of denervation before the neurotoxin, such as a botulinum toxin, begins to exert its therapeutic effect.

A botulinum toxin can be administered by itself or in combination of one or more of the other botulinum toxin serotypes. The botulinum toxin can be a recombinantly made or a hybrid or chimeric botulinum toxin.

My invention also includes within its scope the use of a Clostridial neurotoxin, such as a botulinum toxin, in the preparation of a medicament to assist a dental procedure, by local administration of the Clostridial neurotoxin.

All references, articles, patents, applications and publications set forth above are incorporated herein by reference in their entireties.

Accordingly, the spirit and scope of the following claims should not be limited to the descriptions of the preferred embodiments set forth above.

We claim:

1. A method for assisting a dental procedure, the method comprising the steps of:
(a) administering a botulinum toxin to a mastication muscle of a patient;
(b) waiting a period of time sufficient for the botulinum toxin to weaken and de-program the mastication muscles, so that a force with can be applied by the mastication muscle upon a location where a dental procedure will be conducted in the mouth of the patient is reduced, as compared to a force which can be applied by the mastication muscle at the location of the dental procedure prior to administration of the botulinum toxin, and;
(c) conducting the dental procedure upon the patient.

2. The method of claim 1, wherein the dental procedure is selected from the group consisting of a tooth implantation, a gum tissue transplantation and a dental restoration procedure.

3. The method of claim 1, wherein the botulinum toxin is selected from the group consisting of botulinum toxin types A, B, C, D, E, F and G.

4. The method of claim 1, wherein the botulinum toxin is a botulinum toxin type A.

5. The method of claim 1, wherein the botulinum toxin is administered in an amount of between about 1 unit and about 10,000 units.

6. The method of claim 1, wherein the period of time is between one hour and thirty days.

7. The method of claim 1, wherein the dental procedure is conducted within the mouth of the patient

8. The method of claim 1, wherein the dental procedure is assisted by adherence of a dental item to a substrate in the mouth of the patient.

9. The method of claim 8, wherein the adherence of the dental item occurs in less time, is maintained for a longer period of time, is a stronger adherence and/or occurs with less inflammation, as compared to the adherence of the dental item when a botulinum toxin has not been administered prior to conducting the dental procedure.

10. A method for facilitating an adherence of a dental item to a substrate in the mouth of a patient, the method comprising the steps of:
(a) administering a botulinum toxin to a mastication muscle of a patient;
(b) waiting a period of time sufficient for the botulinum toxin to weaken the mastication muscle, so that a force with can be applied by the mastication muscle upon a location where a dental item can be placed in contact with a substrate in the mouth of the patient is reduced, as compared to a force which can be applied by the mastication muscle at the location where a dental item can be placed in contact with a substrate in the mouth of the patient prior to administration of the botulinum toxin;
(c) placing a dental item in contact with a substrate in the mouth of the patient, and;
(d) observing adherence of the dental item with the substrate in the mouth of the patient, thereby facilitating adherence of the dental item to the substrate in the mouth of the patient.

11. The method of claim 10, wherein the botulinum toxin is selected from the group consisting of botulinum toxin types A, B, C, D, E, F and G.

12. The method of claim 10, wherein the botulinum toxin is a botulinum toxin type A.

13. The method of claim 10, wherein the botulinum toxin is administered in an amount of between about 1 unit and about 10,000 units.

14. The method of claim 10, wherein the period of time is between one hour and thirty days.

15. A method for facilitating adherence of a tooth to a substrate in the mouth of a patient, the method comprising the steps of:
(a) administering a botulinum toxin to a mastication muscle of a patient;
(b) waiting a period of time sufficient for the botulinum toxin to weaken the mastication muscle, so that a force with can be applied by the mastication muscle upon a location where a tooth can be placed in contact with a substrate in the mouth of the patient is reduced, as compared to a force which can be applied by the mastication muscle at the location where the tooth can be placed in contact with a substrate in the mouth of the patient prior to administration of the botulinum toxin;
(c) placing a tooth in contact with a substrate in the mouth of the patient, and;
(d) observing adherence of the tooth with the substrate in the mouth of the patient, thereby facilitating adherence of the tooth to the substrate in the mouth of the patient.

16. The method of claim 15, wherein the botulinum toxin is selected from the group consisting of botulinum toxin types A, B, C, D, E, F and G.

17. A method for accelerating adherence of a transplanted gum tissue to a substrate in the mouth of a patient, the method comprising the steps of:
(a) administering a botulinum toxin to a mastication muscle of a patient;
(b) waiting a period of time sufficient for the botulinum toxin to weaken the mastication muscle, so that a force with can be applied by the mastication muscle upon a location where a gum tissue can be placed in contact with a substrate in the mouth of the patient is reduced, as compared to a force which can be applied by the mastication muscle at the location where the gum tissue can be placed in contact with a substrate in the mouth of the patient prior to administration of the botulinum toxin;
(c) placing a gum tissue in contact with a substrate in the mouth of the patient, and;
(d) observing accelerated adherence of the gum tissue with the substrate in the mouth of the patient, thereby accelerating adherence of the gum tissue to the substrate in the mouth of the patient.

18. The method of claim 17, wherein the botulinum toxin is selected from the group consisting of botulinum toxin types A, B, C, D, E, F and G.

19. A method for obtaining an optimal oral neuromuscular position, the method comprising the steps of:
(a) determining a first bite position of a patient
(b) administering a botulinum toxin to a mastication muscle of the patient which mastication muscle can exert a force which prevents attainment of an optimal oral neuromuscular position, and;
(c) waiting a period of time sufficient for the botulinum toxin to weaken the mastication muscle, thereby obtaining an optimal oral neuromuscular position.

20. The method of claim 19, wherein the botulinum toxin is selected from the group consisting of botulinum toxin types A, B, C, D, E, F and G.
